(19) United States
(12) Patent Application Publication
(10) Pub. No.: US 2026/0034376 A1
(43) Pub. Date: Feb. 5, 2026

(54) SHIFTABLE TRANSDUCER ARRAY WITH ANISOTROPIC MATERIAL LAYER AND FOAM LAYER

(71) Applicant: Novocure GmbH, Baar (CH)

(72) Inventors: Noa HALAVEE, Tel Aviv (IL); Boaz MARSAULT, Haifa (IL); Elie YAACOBI, Haifa (IL); Golan BAR-TAL, Haifa (IL); Nitzan SHANY, Haifa (IL); Yoram WASSERMAN, Haifa (IL); Stas OBUCHOVSKY, Haifa (IL); Nataliya KUPLENNIK, Haifa (IL); David SHAPIRO, Haifa (IL); Dmitry GOLOM, Haifa (IL)

(73) Assignee: Novocure GmbH, Baar (CH)

(21) Appl. No.: 19/289,917

(22) Filed: Aug. 4, 2025

Related U.S. Application Data
(60) Provisional application No. 63/679,495, filed on Aug. 5, 2024.

(51) Int. Cl. A61N 1/40

(52) U.S. Cl. CPC A61N 1/40

(57) ABSTRACT

A transducer apparatus for delivering tumor treating fields to a subject's body, the transducer apparatus including: an array of electrodes, the array configured to be positioned over the subject's body with a front face of the array facing the subject's body, the array comprising electrode elements; an anisotropic material layer electrically coupled to the array of electrodes and located on a front side of the front face of the array, the anisotropic material layer comprising a front face and a back face, the back face facing the array of electrodes, the anisotropic material layer further including a hole passing through the front face and the back face of the anisotropic material layer; and a foam layer coupled to the front face of the anisotropic material layer and covering the hole on the front face of the anisotropic material layer.

### CROSS-REFERENCE TO RELATED APPLICATIONS

This application claims priority to U.S. Provisional Patent Application No. 63/679,495, filed Aug. 5, 2024, which is incorporated herein by reference in its entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into a region of interest by transducers placed on the patient's body and applying alternating current (AC) voltages between the transducers. Conventionally, transducers used to generate TTFields include a plurality of electrode elements comprising ceramic disks. One side of each ceramic disk is positioned against the patient's skin, and the other side of each disc has a conductive backing. Electrical signals are applied to this conductive backing, and these signals are capacitively coupled into the patient's body through the ceramic discs. Conventional transducer designs include rectangular arrays of ceramic disks aligned with each other in straight rows and columns and attached to the subject's body via adhesive.

### BRIEF DESCRIPTION OF THE DRAWINGS

FIGS. 1A and 1B depict a top plan view and a bottom view, respectively, of an example transducer apparatus according to some embodiments.

FIG. 1C depicts a top plan view of an example transducer apparatus according to some embodiments.

FIG. 1D depicts a cross-section view of the example transducer apparatus of FIGS. 1A-1C taken across the section 1D-1D′ according to some embodiments.

FIG. 1E depicts a cross-section view of an example transducer apparatus according to some embodiments.

FIG. 1F depicts a cross-section view of an example transducer apparatus according to some embodiments.

FIGS. 2A and 2B depict a top plan view and a bottom view, respectively, of an example transducer apparatus according to some embodiments.

FIG. 3 is a flowchart depicting an example of applying TTFields to a subject's body according to some embodiments.

Various embodiments are described in detail below with reference to the accompanying drawings, wherein like reference numerals represent like elements, and wherein descriptions of like elements may not be repeated for every embodiment, but may be considered to be the same if previously described herein.

The figures provided herein are for illustrative purposes and may not be to scale. Variations in dimensions, proportions, and configurations may exist between the figures and the actual embodiments. The figures are intended to facilitate understanding of the embodiments and should not be construed as limiting the scope of the disclosure.

### DESCRIPTION OF EMBODIMENTS

This application describes exemplary transducer apparatuses used to apply TTFields to a subject's body, for example, for treating one or more cancers. This application also describes exemplary methods to apply TTFields to a subject's body using transducers.

Transducers used to apply TTFields to a subject's body often include multiple electrode elements electrically coupled together on a substrate and attached to the subject's body at a desired location, for example, via an adhesive backing of the substrate or a separately applied adhesive. Conventional transducers have large, rectangular surfaces so as to maximize a number of electrode elements that are located on the transducer for applying TTFields to the subject's body. However, subjects can experience skin irritation on portions of their skin that are contacted by the electrode elements during TTField treatment. Such irritation may be common at positions directly underneath the electrode elements, where heat and current may be at their highest concentrations, particularly for electrodes around the outer edge of the array.

As recognized by the inventors, on transducer arrays that comprise multiple electrode elements, the portions of the transducer arrays positioned directly beneath the electrode elements may become hotter than the portions of the transducer arrays positioned between the electrode elements. Furthermore, higher currents flow through the electrode elements that may be located along the edge of the array compared to the electrode elements located toward the middle of the array. Further still, an electrode element located at a corner or similar sharp bend in the edge of the array may have a higher current than other electrode elements along the edge and near the center of the array.

As recognized by the inventors, an uneven distribution of current through the transducer array may lead to higher temperature zones (or “hot spots”), e.g., at the corners or edges of the transducer array, which, in turn, may limit the maximum operational current that may be driven by a transducer array and, as a result, the strength of the resulting TTFields.

As recognized by the inventors, a transducer apparatus may also include an anisotropic material layer located on a side of the array of electrode elements facing the subject's body. Such an anisotropic material layer may spread the heat and/or current generated at the individual electrode elements within a plane that is perpendicular to the direction from the electrode elements to the subject's body. Spreading heat and/or current in this plane may reduce the concentration of heat and/or current at locations directly under the individual electrode elements, thus reducing the amount or severity of irritation, if any, that occurs on the subject's skin.

As further recognized by the inventors, a transducer apparatus including an anisotropic material layer may cause irritation to the subject due to the rigidness of the anisotropic material layer. For example, the anisotropic material layer may be relatively inflexible, like a sturdy piece of cardboard, and as such, may not easily conform to a subject's body, which is typically non-planar over a large area or which may bend and change shape as the subject moves. Moreover, due to a non-planar surface on a subject and/or a surface of the subject bending and changing shape, the anisotropic material layer may deform or even crack, resulting in the transducer array producing a less than desired electric field. The inventors have discovered that using a hole in, or substantially in, the center or the middle of the anisotropic material layer may help to alleviate this problem.

As further recognized by the inventors, a transducer apparatus including an anisotropic material layer may cause irritation to the subject due to the sharp edges of the anisotropic material layer. When a piece of anisotropic material is cut to size to be used as an anisotropic material layer in a transducer apparatus, the edges of the transducer apparatus may be sharp. When these edges come in contact with a subject, the subject may experience discomfort. To reduce this discomfort from the transducer apparatus, the subject may inadvertently place the transducer apparatus in a different, non-desired position on the subject for delivery of TTFields. This may result in the subject receiving less than a desired dosage of TTFields. Further, due to this discomfort from the transducer apparatus, the subject may decrease usage of the transducer apparatus, which may result in the subject receiving less than a desired dosage of TTFields. The inventors have discovered that using a foam layer with the anisotropic material layer may help to alleviate this discomfort experienced by a subject. The inventors have further discovered that the foam layer may advantageously provide some additional flexibility for the array when the subject moves or twists the body part associated with the location of the array. Further, the added flexibility is enhanced when the foam layer covers (on the skin-facing side) a central hole in the anisotropic material layer.

Descriptions of embodiments related to specific exemplary drawings herein may be applicable, and may be combined with, descriptions of embodiments related to other exemplary drawings herein unless otherwise indicated herein or otherwise clearly contradicted by context.

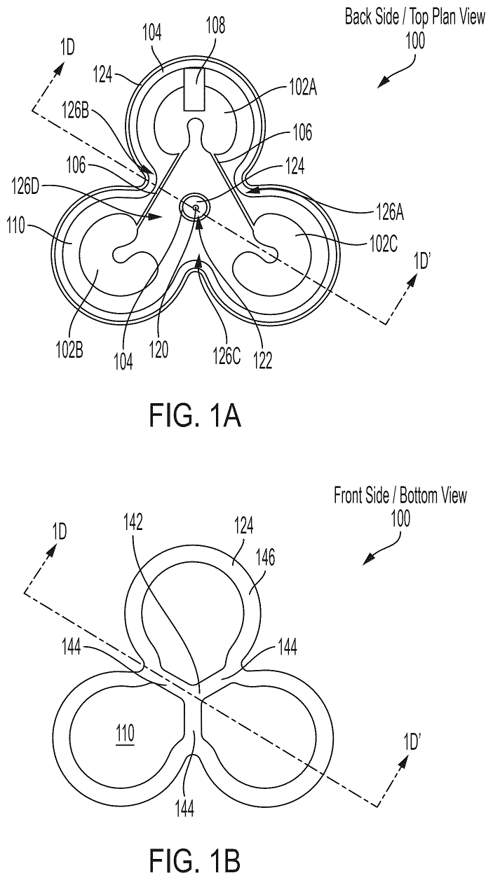

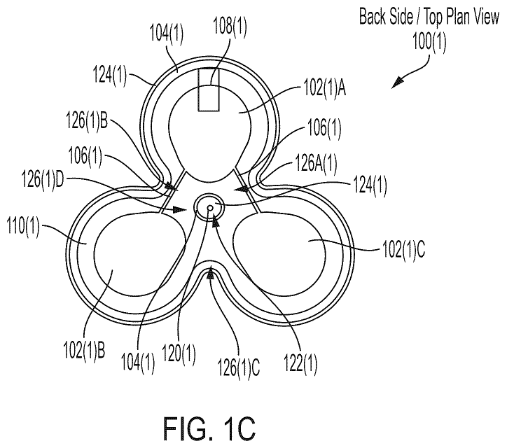

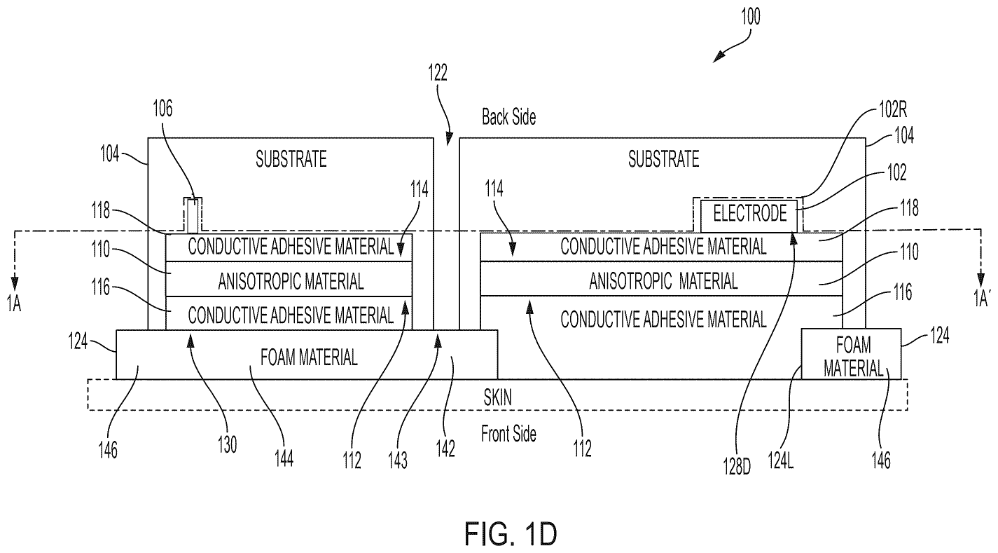

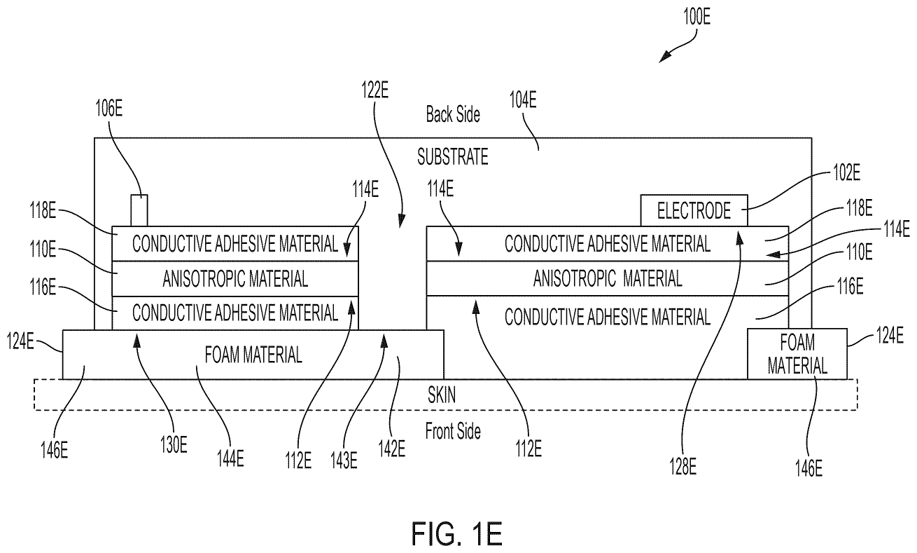

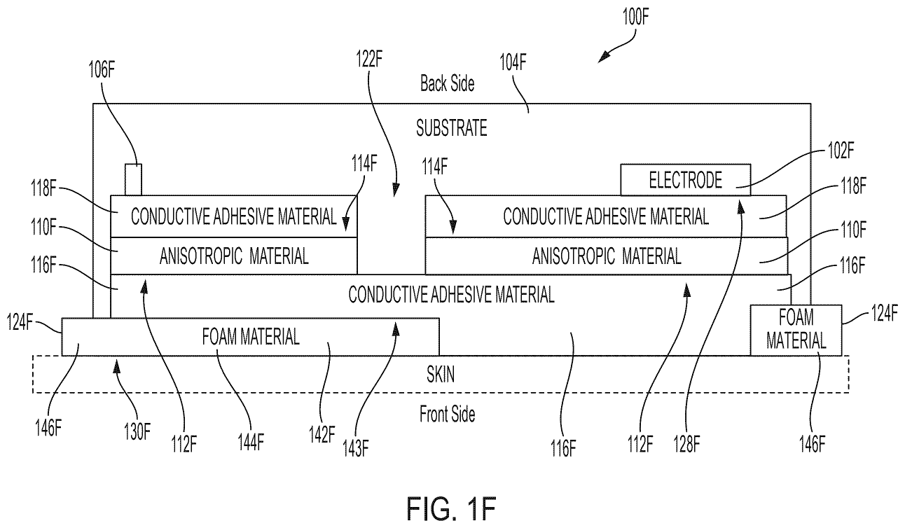

FIGS. 1A and 1B depict a top plan view and a bottom view, respectively, of an example transducer apparatus 100 according to some embodiments. FIG. 1D depicts a cross-section view of the example transducer apparatus 100 of FIGS. 1A and 1B taken across the section 1D-1D′ according to some embodiments. FIGS. 1A and 1B illustrate the transducer apparatus 100 as viewed from a direction perpendicular to a face of the transducer apparatus 100. FIG. 1A illustrates the back side of the transducer apparatus 100 (i.e., the side facing away from the subject's body). FIG. 1B illustrates the front side of the transducer apparatus 100 (i.e., the side facing the subject's body). Further, FIG. 1A is a plan view of the transducer apparatus 100 along line 1A-1A′ of FIG. 1D, and as such, only a portion of the substrate 104 is depicted in FIG. 1A.

As shown in FIGS. 1A and 1D, the transducer apparatus 100 may have a plurality of paddle shapes extending from a center, or have a shape comprised of a plurality of substantially circular shapes connected about a center. The transducer apparatus 100, as illustrated, may include a plurality of electrode elements 102 (i.e., 102A, 102B, 102C), a substrate 104, and an anisotropic material layer 110 (FIG. 1A). The transducer apparatus 100 may include one or more blank spaces 126 (i.e. 126A-D), which do not overlap with any of the electrode elements 102. At least part of one or more of the blank spaces 126 may be a relief region, defined herein as either: (1) void regions of the transducer apparatus 100 that are fully uncovered or fully uncovered other than the transducer substrate and/or an anisotropic material layer (with or without conductive adhesive layer(s) (e.g., 116, 116E, 116F) and/or a conductive layer (e.g., 118, 118E, 118F) (FIG. 1D)); (2) non-adhesive regions comprising a medication substrate capable of receiving, absorbing, and/or holding a topical medication applied thereto; or (3) medication regions of the transducer apparatus comprising a medication substrate and a topical medication integrated therein or thereon used to administer a topical medication to an area of the subject's skin. These relief regions may, optionally, have no exposed adhesive present.

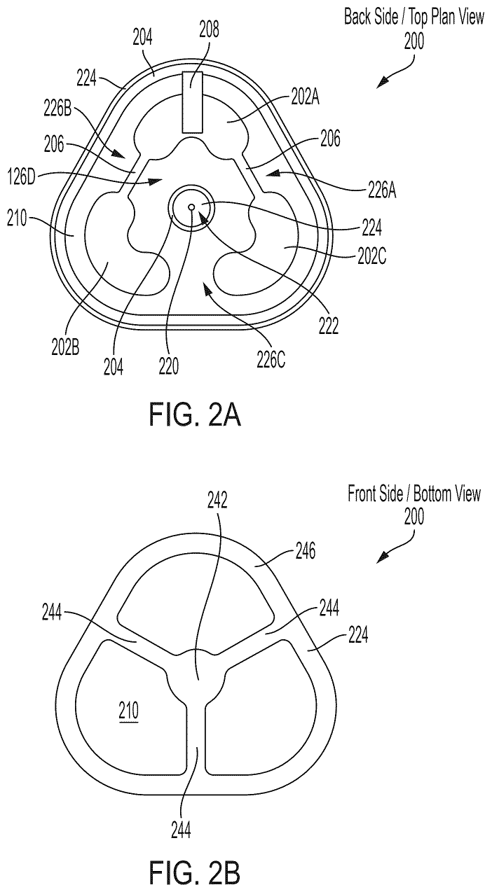

The plurality of electrodes elements 102 may be spaced about a centroid 120 of the transducer apparatus 100, and the blank spaces 126 may each be located between two adjacent electrodes and around the centroid 120. In some embodiments, the transducer apparatus 100 may have an alternating pattern of electrodes 102 and blank spaces 126. In other embodiments, non-alternating rotational patterns of electrodes 102 and blank spaces 126 may be used. The electrodes 102 may be electrically coupled together via one or more printed circuit board (PCB) connector(s) 106 or wire(s). The transducer apparatus may be electrically coupled to a voltage generator via a lead connector 108. The PCB connector(s) 106 and lead connector 108 (and 206, 208, respectively, in FIG. 2A) are not electrodes and are non-adhesive regions. Although three electrodes 102 and four blank spaces 126 are shown in FIGS. 1A and 1B, other embodiments may include different numbers of electrodes 102, blank spaces 126, or both in the array. For example, some embodiments may include six electrodes 102 and seven blank spaces 126; or five electrodes 102 and six blank spaces 126; or four electrodes 102 and five blank spaces 126; or three electrodes 102 and four blank spaces 126; or two electrodes 102 and three blank spaces 126.

As shown in FIG. 1A, each electrode 102 of the array may be u-shaped, substantially u-shaped, jelly-bean or kidney-bean shaped, substantially jelly-bean or kidney-bean shaped, horseshoe shaped, or substantially horseshoe shaped. In some embodiments, the end-points of the u-shape may be close together. In other embodiments, the end-points of the u-shape may have a gap in between. In some embodiments, the open end of the u-shaped electrode 102 may face the centroid 120 of the transducer apparatus 100. In addition, a centroid of each electrode 102 may be spaced substantially equidistant from the centroid 120 of the transducer apparatus 100. Each electrode 102 may have a substantially similar shape The electrodes 102 may be spaced substantially equidistant from each other about the centroid 120 of the array. The electrodes 102 may be spaced substantially equidistant from the centroid 120 of the array and/or may be spaced substantially equidistant from each other.

The transducer apparatus 100 may include an anisotropic material layer 110 directly or indirectly electrically coupled to the plurality of electrodes 102 and located on a front face 128 (128D in FIG. 1D) of the electrodes 102 configured to face the subject's body. The anisotropic material layer 110 may take any of the forms and include any of the features described in further detail below with reference to the anisotropic material layer 110, 110E, 110F of FIGS. 1D-1F.

The anisotropic material layer 110 may be disposed over the plurality of electrodes such that the anisotropic material layer 110 covers the electrode elements 102A-C and, optionally, the at least one blank space 126 (e.g., void space) in the array. In some embodiments, the anisotropic material layer 110 may be disposed over the plurality of electrodes to cover the electrode elements 102A-C and blank space 126A-D in the array. In some embodiments, the anisotropic material layer 110 may not extend outward all the way to the edge of the substrate layer 104. The anisotropic material layer 110 may include a hole 122 in, or substantially in, the center or the middle of the anisotropic material layer 110, passing through the front face 112 (112E, 112F in FIGS. 1E and 1F) and the back face 114 (114E, 114F in FIGS. 1E and 1F), facilitating flexibility and pliability of the anisotropic material layer 110. The hole 122 may be circular, oval, or ovoid, or substantially circular oval, or ovoid in shape. The hole may be positioned at or near the centroid 120 of the transducer apparatus 100. For example, the centroid 120 of the transducer apparatus 100 may be positioned at the center of the hole 122. Furthermore, the centroid of each electrode 102 may be spaced substantially equidistant from the hole 122. The PCB connector(s) 106 or wire(s) may not pass over the hole 122 of the anisotropic material layer 110 and/or may be positioned equidistantly from the hole 122 of the anisotropic material layer 110.

The transducer apparatus 100 may further include a foam material layer 124 directly or indirectly coupled to the anisotropic material layer 110 and located on a front face 112 of the anisotropic material layer 110 configured to contact the subject's body. The foam material layer 124 may cover at least the hole 122 formed in the anisotropic material layer 110. In some embodiments, the foam material layer 124 may include a hole covering portion 142, positioned over the hole 122 in the anisotropic material layer 110 (FIG. 1D). The hole covering portion 142 may be dimensioned larger than the hole 122 in the anisotropic material layer 110. The foam material layer may include a perimeter portion 146 covering a perimeter of the anisotropic material layer 110. In some embodiments, the anisotropic material layer 110 may not extend outward all the way to the edge of the foam material layer 124. In some embodiments, the perimeter portion 146 of the foam material layer 124 may cover up to the edge of the perimeter of the anisotropic material layer 110. The perimeter of the perimeter portion of the foam material layer 124 may be larger than the perimeter of the anisotropic material layer 110.

As further shown in FIG. 1B, the foam material layer 124 may cover at least the hole 122 on the side of the transducer apparatus 100 facing the subject's body. The perimeter of the perimeter portion 146 of the foam material layer 124 may be larger than the perimeter of the anisotropic material layer 110. The perimeter of the foam material layer 124 may have substantially the same shape as the perimeter of the anisotropic material layer 110. The foam material layer 124 may include at least one connection portion 144 connecting the hole covering portion 142 and the perimeter portion 146. The at least one connection portion 144 may cover the front side 112 of the anisotropic material layer 110. When viewed from a direction perpendicular to the front face 112 of the anisotropic material layer 110, the at least one connection portion 144 may be located coincident with the blank spaces 126, except on the skin-facing side of the anisotropic material layer 110. The foam material layer 124 may cover at most 30%, or at most 40%, or at most 50%, of the anisotropic material layer 110.

FIG. 1C depicts a top plan view of an example transducer apparatus 100(1) according to some embodiments. The transducer apparatus 100(1) in FIG. 1C is the same as the transducer apparatus 100 in FIG. 1A, except for the shape of the electrode elements. The parts in FIG. 1C use the same reference numbering as the parts in FIG. 1A, except for the added suffix of “(1)”. In FIG. 1C, the electrode elements 102(1) (i.e., 102(1)A, 102(1)B, 102(1)C) are ovoid or substantially ovoid shaped. In some embodiments, the electrode elements 102(1) may have a different shape, such as oval shaped or triangular or substantially triangular shaped, or rounded triangular or substantially rounded triangular shaped.

FIG. 1D depicts a cross-section view of the example transducer apparatus 100 of FIGS. 1A and 1B taken across the section 1D-1D′ according to some embodiments.

The substrate 104 may be configured for attaching a front side of the transducer 100 to a subject's body. As discussed above, since FIG. 1A is a plan view of the transducer apparatus 100 along line 1A-1A′ of FIG. 1D, only a portion of the substrate 104 is depicted in FIG. 1A. However, as illustrated in FIG. 1D, the substrate 104 may substantially cover the back side of the transducer apparatus 100. The substrate 104 may be located on the back side of the plurality of electrode elements 102. The substrate 104 may be configured to hold the transducer array 100 against the subject's body. Suitable materials for the substrate 104 include, for example, cloth, foam, flexible plastic, and/or a conductive medical gel. The transducer 100 may be affixed to the subject's body via the substrate 104 (e.g., via an adhesive layer and/or a conductive medical gel). The substrate may be an adhesive bandage. The adhesive layer that contacts the subject's skin may be present around the outer perimeter of the array of electrodes, and/or may be present in a central-middle area defined by the electrodes (or between one or more gaps between electrodes).

The transducer apparatus 100 may include the hole 122 that extends through each layer of the transducer apparatus except for the foam layer 124. The transducer apparatus 100 includes an anisotropic material layer 110. As shown, the anisotropic material layer 110 has a front face 112 and a back face 114, wherein the back face 114 faces the array of electrode elements 102. The anisotropic material layer 110 has anisotropic thermal properties and/or anisotropic electrical properties. If the anisotropic material layer 110 has anisotropic thermal properties (for example, greater thermal conductivity in the plane of the layer than through the plane of the layer), then the layer spreads the heat out more evenly over a larger surface area. If the anisotropic material layer 110 has anisotropic electrical properties (for example, greater electrical conductivity in the plane of the layer than through the plane of the layer), then the layer spreads the current out more evenly over a larger surface area. In each case, this lowers the temperature of the hot spots and raises the temperature of the cooler regions when a given AC voltage is applied to the array of electrode elements. Accordingly, the current may be increased (thereby increasing the therapeutic effect) without exceeding the safety temperature threshold at any point on the subject's skin.

In some embodiments, the anisotropic material layer 110 is anisotropic with respect to electrical conductivity properties. In some embodiments, the anisotropic material layer 110 is anisotropic with respect to thermal conductivity properties. In some embodiments, the anisotropic material layer 110 is anisotropic with respect to both electrical conductivity properties and thermal conductivity properties.

The anisotropic thermal properties include directional thermal properties. Specifically, the anisotropic material layer 110 may have a first thermal conductivity in a direction that is perpendicular to its front face (skin-facing surface) 112 that is different from a thermal conductivity of the anisotropic material layer 110 in directions that are parallel to the front face 112. For example, the thermal conductivity of the anisotropic material layer 110 in directions parallel to the front face 112 may be more than two times higher than the first thermal conductivity. In some preferred embodiments, the thermal conductivity in the parallel directions may be more than ten times higher than the first thermal conductivity. For example, the thermal conductivity of the sheet in directions that are parallel to the front face 112 may be more than: 1.5 times, 2 times, 3 times, 5 times, 10 times, 20 times, 100 times, 200 times, or even more than 1,000 times higher than the first thermal conductivity.

The anisotropic electrical properties include directional electrical properties. Specifically, the anisotropic material layer 110 may have a first electrical conductivity (or, conversely, resistance) in a direction that is perpendicular to its front face 112 that is different from an electrical conductivity (or resistance) of the anisotropic material layer 110 in directions that are parallel to the front face 112. For example, the resistance of the anisotropic material layer 110 in directions parallel to the front face 112 may be less than the first resistance. In some preferred embodiments, the resistance in the parallel directions may be less than half of the first resistance or less than 10% of the first resistance. For example, the resistance of the anisotropic material layer 110 in directions that are parallel to the front face 112 may be less than: 75%, 50%, 40%, 30%, 20%, 10%, 5%, 1%, 0.5%, or even less than 0.1% of the first resistance.

In some embodiments (e.g., when the anisotropic material layer 110 is a sheet of pyrolytic graphite), the anisotropic material layer 110 has both anisotropic electrical properties and anisotropic thermal properties.

The anisotropic material layer 110 may comprise graphite (e.g., a sheet of graphite or a graphite sheet). Examples of suitable forms of graphite include synthetic graphite, such as pyrolytic graphite (including, but not limited to, Pyrolytic Graphite Sheet (PGS), available from Panasonic Industry, Kadoma, Osaka, Japan), other forms of synthetic graphite, including but not limited to, graphite foil made from compressed high purity exfoliated mineral graphite (including, but not limited to, that supplied as MinGraph® 2010A Flexible Graphite, available from Mineral Seal Corp., Tucson, Arizona, USA), or graphitized polymer film, e.g., graphitized polyimide film, (including, but not limited to, that supplied by Kaneka Corp., Moka, Tochigi, Japan). In alternative embodiments, conductive anisotropic materials other than graphite may be used instead of graphite.

In some embodiments, the anisotropic material layer 110 may be a sheet of pyrolytic graphite. Thermal conductivity of pyrolytic graphite sheets in directions that are parallel to the front face 112 of those sheets is typically more than 50 times higher than the thermal conductivity of those sheets in directions that are perpendicular to the front face 112. Electrical resistivity of pyrolytic graphite sheets in directions that are parallel to the front face 112 of those sheets is typically less than 2% of the electrical resistivity of those sheets in directions that are perpendicular to the front face 112.

While an anisotropic material layer comprised of graphite may reduce the concentration of heat and/or current at locations directly under the individual electrode elements—thus reducing the amount or severity of irritation, if any, that occurs on the subject's skin—such anisotropic material may be relatively inflexible. In other words, when pressed against a non-flat surface, the anisotropic material layer may be at risk of cracking and/or breaking. As such, the anisotropic material layer may not easily conform to a subject's body, which is typically non-planar over a large area or which may bend and change shape as the subject moves. Additionally, due to a non-planar surface on a subject and/or a surface of the subject bending and changing shape, the anisotropic material layer may deform or even crack, resulting in the transducer array producing a less than desired electric field. Obtaining a flush contact surface between the electrode element and the subject's skin may improve the effectiveness of the treatment. Accordingly, the anisotropic material layer 110 includes the hole 122 substantially in the center of the anisotropic material layer 110, passing through the front face 112 and the back face 114, facilitating flexibility and pliability of the anisotropic material layer 110. The hole 122 in the anisotropic material layer 110 further facilitates generation of an effective electric field and obtaining a flush contact between the transducer 100 and the subject's skin.

The transducer 100 may further include at least one layer of conductive adhesive material 116 disposed on a front facing side of the anisotropic material layer 110. In some embodiments, the at least one layer of conductive adhesive material 116 may be disposed on the front face 112 of the anisotropic material layer 110. The conductive adhesive material 116 may include the hole 122 and may be aligned with the hole 122 through the anisotropic material layer 110. The at least one layer of conductive adhesive material 116 may have a biocompatible front surface. In some embodiments, there may only be a single layer of conductive adhesive material 116, and that single layer (the front layer) may be biocompatible. In some embodiments, there may be more than one layer of conductive adhesive material 116, in which case only the front layer may be biocompatible, or the front layer and one or more other layers may be biocompatible. In some embodiments, the front layer of conductive adhesive material 116 may be configured to ensure good electrical contact between the device and the body. In some embodiments, the front layer of conductive adhesive material 116 may be configured to ensure good adhesion between the anisotropic material layer 110 and the foam material layer 124. In some embodiments, the front layer of conductive adhesive material 116 may cover the entire front face 112 of the anisotropic material layer 110. The front layer of conductive adhesive material 116 may be the same size or larger than the anisotropic material layer 110. In some embodiments, the front layer of conductive adhesive material 116 may comprise hydrogel. In these embodiments, the hydrogel may have a thickness between 50 μm and 2,000 μm. In some embodiments, the front layer of conductive adhesive material 116 may comprise a conductive adhesive composite as further disclosed herein.

The transducer 100 may further include at least one conductive material layer. A conductive material layer 118 may be positioned between the array of electrode elements 102 and the back face 114 of the anisotropic material layer 110 facing the array. The conductive material layer 118 may facilitate the electrical contact between the array of electrode elements 102 and the back face 114 of the anisotropic material layer 110. The conductive material layer 118 may include the hole 122 and may be aligned with the hole 122 through the anisotropic material layer 110 and the conductive adhesive material 116. In some embodiments, the conductive material layer 118 may be a layer of hydrogel. In other embodiments, a different conductive material (e.g., conductive grease, conductive adhesives, conductive tape, etc.) may be used. For example, the conductive material layer 118 may comprise a conductive adhesive composite as further disclosed herein.

In some embodiments, the at least one layer of conductive adhesive material 116 and/or the layer of conductive material 118 may be a single layer of non-hydrogel conductive adhesive such as the developmental product FLX068983-FLEXcon® OMNI-WAVE™ TT 200 BLACK H-502 150 POLY H-9 44PP-8 from FLEXcon, Spencer, MA, USA, or other such OMNI-WAVE products from FLEXcon; or ARcare® 8006 electrically conductive adhesive composition manufactured and sold by Adhesives Research, Inc. (Glen Rock, PA, USA). Non-hydrogel conductive adhesives may comprise a waterless polymer with adhesive properties and carbon particles, powder, fibers, flakes, granules and/or nanotubes. The adhesive polymer may be, for example, an acrylic polymer or a silicone polymer, or combination thereof. In some embodiments, the acrylic polymer or silicone polymer (and composites therefrom) may not be tacky, or may be minimally tacky, and yet may have adhesive properties in that the polymer may still aid in adhering the array to the skin. Herein, such materials may also be conductive adhesive materials. In some embodiments, the acrylic polymer or silicone polymer (or respective composites) may be available as acrylic- or silicone-based carbon-filled adhesive tapes. The adhesive may additionally include one or more conductive polymers (such as, for example, polyaniline (PANI), or poly(3,4-ethylenedioxythiophene) (PEDOT), or others known in the art). The conductive filler in the at least one layer of conductive adhesive material 116 or conductive material 118 may be non-metallic. In these embodiments, the conductive adhesive may have a thickness between 10 and 2,000 μm, such as, from 20 to 1,000 μm, or 30 to 400 μm.

In some embodiments, the transducer 100 may be constructed using a pre-formed 3- (or more) layer laminate comprising the conductive material 118, the anisotropic material layer 110, and the at least one layer of conductive adhesive material 116. In some embodiments, the at least one conductive adhesive material 116 and the conductive material 118 may both be conductive adhesive composites as described above, and the anisotropic material layer 110 may be a thin sheet of synthetic graphite such as pyrolytic graphite, as described above. In some embodiments, the at least one conductive adhesive material 116 and the conductive material 118 may be the same material or may be different. By way of example, in some embodiments, both the conductive adhesive material 116 and the conductive material 118 may comprise an acrylic polymer and a carbon powder filler; or both the conductive adhesive material 116 and the conductive material 118 may comprise an acrylic polymer and a carbon fiber filler. In some embodiments, the conductive adhesive material 116 may comprise an acrylic polymer and a carbon fiber filler, and the conductive material 118 may comprise an acrylic polymer and a carbon powder filler; or vice-versa. In some embodiments, one or both of the conductive adhesive material 116 and the conductive material 118 may be a hydrogel.

The substrate 104 may also include the hole 122 and may be aligned with the hole 122 through the anisotropic material layer 110, the conductive adhesive material 116, and the conductive material 118, such that the hole 122 extends through each layer located in the same position. The hole 122 may be in the center or substantially in the center of the transducer apparatus 100. In some embodiments, the hole 122 may be circular, oval, or ovoid shaped, or substantially circular, oval, or ovoid shaped. In some embodiments, the hole 122 may be at least 0.5 cm and at most 3.0 cm in diameter. For example, the hole 122 may be 0.5 cm, 1.0 cm, 1.5 cm, 2.0 cm, 2.5 cm, or 3.0 cm in diameter, or may range in diameter between any of these listed diameters. In some embodiments, the hole 122 may be at least 0.5% to at most 5.0% of the area of the anisotropic material layer 110. For example, the hole 122 may be 0.5%, 1.0%, 1.5%, 2.0%, 2.5%, 3.0%, 3.5%, 4.0%, 4.5%, or 5.0% of the area of the anisotropic material layer 110, or may range between any of these listed percentages. In some embodiments, the hole 122 may be at least 0.5% to at most 10%, or at most 20%, or at most 30%, or at most 40%, or at most 50%, of the area of the anisotropic material layer 110. In some embodiments, the hole 122 may be at least 0.5%, or at least 10%, or at least 20%, or at least 30%, or at least 40%, to at most 50%, of the area of the anisotropic material layer 110. In some embodiments, the hole in the substrate 104 may have a diameter smaller than the hole 122 of the anisotropic material layer 110. In some embodiments, the hole in the substrate 104 may have a diameter greater than the hole 122 of the anisotropic material layer 110. In some embodiments, the hole in the substrate 104 may match the size, shape, and location of the hole in the anisotropic material layer 110.

The transducer 100 may further include at least one foam material layer 124. The foam material layer 124 may be positioned between a front face 130 of the conductive adhesive material 116 and the subject's body. A back face 143 of the hole covering portion 142 of the foam material layer 124 may not be in contact with any other part or portion of the transducer apparatus 100. By being free of contact on the back face 143, the transducer apparatus 100 may be more pliable against a subject's body.

The foam material layer 124 may facilitate a comfortable surface against the subject's body. In some embodiments, a transducer 100 having the anisotropic material layer 110 may include the foam material layer 124 to provide a conformable material in contact with the subject's body. The foam material layer 124 may be in the same plane as the conductive adhesive layer 116 so that the electrode element 102 is placed as close as possible to the subject's skin, without the area of the electrode element 102 of the transducer apparatus 100 projecting past the foam material layer 124. For example, as shown in FIG. 1D, the right side 102R of the electrode 102 is to the left of the left side 124L of the foam material layer 124.

In some embodiments, the foam material layer 124 may be formed at least partially of a soft material with airy open space. In some embodiments, the foam material layer 124 may be formed of a non-porous material to improve sterilizability and cleanability. In some embodiments, the foam material layer 124 may comprise one or more of low-density polyethylene (LDPE), silicone, polyurethane, or ethylene-vinyl acetate (EVA) closed-cell foam.

The transducer apparatus 100 may comprise an array of substantially flat electrode elements 102. The array of electrode elements 102 may or may not be capacitively coupled. The electrode elements 102 may be non-ceramic dielectric materials positioned over a plurality of flat conductors such as, for example, polymer films disposed over pads on a printed circuit board or over flat pieces of metal. In some embodiments, the electrode elements 102 may be ceramic elements. In some embodiments, the electrode elements may not have a dielectric material.

In some embodiments, the dielectric material of the electrode elements 102 may have a dielectric constant ranging from 10 to 50,000. In some embodiments, the layer of dielectric material may comprise a high dielectric polymer material such as poly(vinylidene fluoride-trifluoroethylene-chlorotrifluoroethylene) and/or poly(vinylidene fluoride-trifluoroethylene-1-chlorofluoroethylene). Those two polymers are abbreviated herein as “Poly(VDF-TrFE-CTFE)” and “Poly(VDF-TrFE-CFE),” respectively. The dielectric constant of these materials may be on the order of 40. In some embodiments, the polymer layer may be poly(vinylidene fluoride-trifluoroethylene-chlorotrifluoroethylene-chlorofluoroethylene) or “Poly(VDF-TrFE-CTFE-CFE).”

In some embodiments, the layer of dielectric material of the electrode elements 102 may comprise a terpolymer comprising polymerized units of monomers such as VDF, TrFE, CFE and/or CTFE in any suitable molar ratio. Suitable terpolymers may include those, for example, having 30 to 80 mol % VDF, 5 to 60 mol % TrFE, with CFE and/or CTFE constituting the balance of the mol % of the terpolymer.

FIG. 1E depicts a cross-section view of an example transducer apparatus 100E according to some embodiments. The transducer apparatus 100E in FIG. 1E is the same as the transducer apparatus 100 in FIG. 1D, except for the substrate. The parts in FIG. 1E use the same reference numbering as the parts in FIG. 1D, except for the added suffix of “E”. In FIG. 1E, the substrate 104E fills in the hole 122E in the anisotropic material layer 110E. In some embodiments, the substrate 104E may be in contact with the back face 143E of the foam material layer 124E. In some embodiments, the substrate 104E may be in contact with the back face 143E of the hole covering portion 142E of the foam material layer 124E.

FIG. 1F depicts a cross-section view of an example transducer apparatus 100F according to some embodiments. The transducer apparatus 100F in FIG. 1F is the same as the transducer apparatus 100E in FIG. 1E, except for the conductive adhesive material layer 116F. The parts in FIG. 1F use the same reference numbering as the parts in FIG. 1D, except for the added suffix of “F”. In FIG. 1F, the conductive adhesive material layer 116F fills in the hole 122F on the front side 112F of the anisotropic material layer 110F. In some embodiments, the conductive adhesive material layer 116F may be in contact with the back face 143F of the foam material layer 124F. In some embodiments, the conductive adhesive material layer 116F may be in contact with the back face 143F of the hole covering portion 142F of the foam material layer 124F.

FIGS. 2A and 2B depict a top plan view and a bottom view, respectively, of an example transducer apparatus 200 according to some embodiments. The transducer apparatus 200 in FIGS. 2A and 2B is substantially the same as the transducer apparatus 100 in FIGS. 1A, 1B, and 1C, except for the shape of the transducer apparatus, the shape of the electrodes, and the shape of the foam material layer. The Figure labelling follows the same convention as that used in FIGS. 1A, 1B, and 1C. As seen in FIGS. 2A and 2B, the transducer apparatus 200 may include a plurality of electrode elements 202 (202A, 202B, 202C) positioned on a substrate 204. As shown, the electrodes 202 may be disposed on an anisotropic material layer 210. Additionally, the electrodes 202 may be arranged substantially equidistant around the centroid 220 of the transducer apparatus 200. The transducer apparatus may comprise a conductive adhesive material layer 216 on a front face 212 of the anisotropic material layer 210, a conductive material layer 218, such as a conductive adhesive, on a back face of the anisotropic material layer 210, and a foam material layer 224. The blank spaces 226A-D, PCB connector(s) 206, and lead connector 208 are analogous to 126A-D, 106, and 108 in FIG. 1A. As shown in FIGS. 2A and 2B, the transducer apparatus 200 may be substantially asymmetric oval, -ovaloid, -ovoid, rounded triangular, or trilobal in shape. As shown in FIGS. 2A and 2B, the electrode elements 202 may have a c-shape, a substantially c-shape, a jelly-bean or kidney bean shape, a substantially jelly-bean or kidney bean shape, a bent elbow shape, or a substantially bent elbow shape. As shown in FIG. 2B, the hole covering portion 242 of the foam material layer 224 may have a shape to match a shape of the hole 222. The shape of the hole covering portion 242 may mirror the shape of and/or may be larger than the shape of the hole 222. The connecting portion 244 and perimeter portion 246 of the foam material layer 224 is analogous to the connecting portion 144 and perimeter portion 146 of the foam material layer 124 in FIG. 1B.

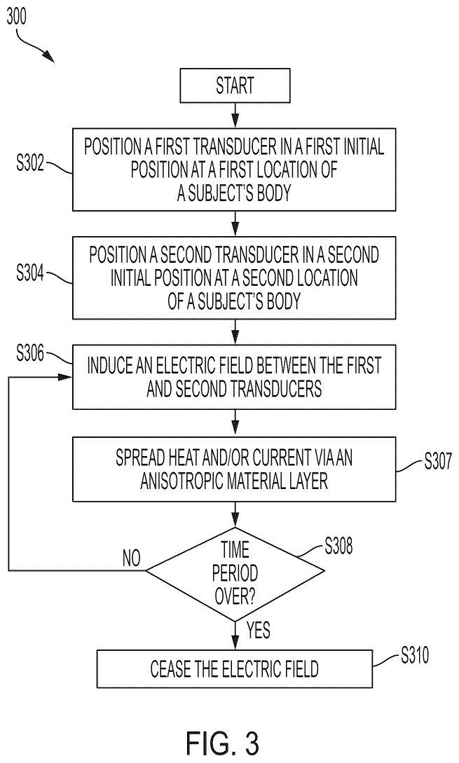

FIG. 3 depicts an example method 300 of applying TTFields to a subject's body in accordance with the present techniques. In some embodiments, TTFields may include low-intensity (e.g., about 1 V/cm to about 10 V/cm) alternating electric fields of medium frequencies (e.g., about 50 kHz to about 1 MHz, and, for some embodiments, about 100 kHz to about 300 kHz) that when applied to a conductive medium, such as a human body, via electrodes may be used, for example, to treat tumors as described in U.S. Pat. Nos. 7,016,725, 7,089,054, 7,333,852, 7,565,205, 7,805,201, and 8,244,345 by Palti and in Eilon D. Kirson et al., “Disruption of Cancer Cell Replication by Alternating Electric Fields,” Cancer Res. 2004 64:3288-3295; all of which are hereby incorporated by reference in their entirety.

The method 300 begins at step S302 with positioning a first transducer in a first initial position at a first location of the subject's body. The first transducer may be one of the example transducer apparatuses discussed herein. The first transducer may be affixed to the subject's body via an adhesive layer. The first transducer may include an anisotropic material layer electrically coupled to the plurality of electrodes and located between the plurality of electrodes and the subject's body, with the anisotropic material layer having a hole cutout therein, the hole being located substantially in the center of the anisotropic material layer, adjacent electrodes.

At step S304, the method 300 may include positioning a second transducer in a second initial position at a second location of the subject's body. The second transducer may be one of the example transducer apparatuses discussed herein. The second transducer may be affixed to the subject's body via an adhesive layer. The second transducer may include an anisotropic material layer electrically coupled to the plurality of electrodes and located between the plurality of electrodes and the subject's body, with the anisotropic material layer having a hole cutout therein, the hole being located substantially in the center of the anisotropic material layer, adjacent electrodes.

At step S306, the method 300 may include inducing an electric field between the first transducer located at the first location of the subject's body and the second transducer located at the second location of the subject's body.

At step S307, during inducing the electric field, the method 300 may include spreading heat and/or current via an anisotropic material layer from the plurality of electrodes in a plane perpendicular to a direction from the plurality of electrodes to the subject's body.

At step S308, the method 300 may include determining whether a first period of time has passed. Upon determining that the first period of time has passed, the method 300 proceeds to step S310. Otherwise, the method 300 returns to step S306. After inducing the electric field for more than the first period of time, the method 300 proceeds to step S310, which may include ceasing the electric field.

The invention includes other illustrative embodiments (“Embodiments”) as follows.

### Embodiment 1

A transducer apparatus for delivering tumor treating fields to a subject's body, the transducer apparatus comprising: an array of electrodes, the array configured to be positioned over the subject's body with a front face of the array facing the subject's body, the array comprising electrode elements; an anisotropic material layer electrically coupled to the array of electrodes and located on a front side of the front face of the array, the anisotropic material layer comprising a front face and a back face, the back face facing the array of electrodes, the anisotropic material layer further comprising a hole passing through the front face and the back face of the anisotropic material layer; and a foam layer coupled to the front face of the anisotropic material layer and covering at least the hole on the front face of the anisotropic material layer.

### Embodiment 1A

The transducer apparatus of Embodiment 1, wherein the anisotropic material layer is electrically coupled to the array of electrodes and is located on the front face of the array of electrodes.

### Embodiment 2

The transducer apparatus of Embodiment 1, wherein when viewed from the front face of the anisotropic material layer, the foam layer covers at most 50% of the area of the anisotropic material layer.

### Embodiment 2A

The transducer apparatus of Embodiment 1, wherein when viewed from the front face of the anisotropic material layer, the foam layer comprises a hole covering portion to cover the hole on the front face of the anisotropic material layer.

### Embodiment 3

The transducer apparatus of Embodiment 1, wherein when viewed from the front face of the anisotropic material layer, the foam layer comprises a hole covering portion to cover the hole on the front face of the anisotropic material layer and the hole covering portion is larger than the hole on the front face of the anisotropic material layer.

### Embodiment 4

The transducer apparatus of Embodiment 1, wherein when viewed from the front face of the anisotropic material layer, the foam layer comprises a hole covering portion to cover the hole on the front face of the anisotropic material layer and the foam layer further comprises a perimeter portion covering a perimeter of the anisotropic material layer.

### Embodiment 5

The transducer apparatus of Embodiment 4, wherein when viewed from the front face of the anisotropic material layer, the foam layer further comprises at least one connecting portion connecting the hole covering portion and the perimeter portion, wherein the at least one connecting portion covers the anisotropic material layer.

### Embodiment 6

The transducer apparatus of Embodiment 1, wherein when viewed from the back face of the anisotropic material layer, a perimeter of the foam layer is larger than a perimeter of the anisotropic material layer.

### Embodiment 7

The transducer apparatus of Embodiment 6, wherein when viewed from the back face of the anisotropic material layer, a shape of the perimeter of the foam layer is the same as or substantially the same as a shape of the perimeter of the anisotropic material layer.

### Embodiment 8

The transducer apparatus of Embodiment 1, wherein the hole is centrally located in the front face and the back face of the anisotropic material layer.

### Embodiment 9

The transducer apparatus of Embodiment 1, wherein the hole is circular, oval, ovoid, or substantially circular, oval, ovoid in shape.

### Embodiment 10

The transducer apparatus of Embodiment 1, wherein the electrode elements are positioned around the hole of the anisotropic material layer.

### Embodiment 11

The transducer apparatus of Embodiment 1, wherein the electrode elements are positioned equidistantly from each other and equidistantly from the hole of the anisotropic material layer.

### Embodiment 12

The transducer apparatus of Embodiment 1, further comprising at least one layer of conductive adhesive material or hydrogel located on a front facing side of the anisotropic material layer.

### Embodiment 12A

The transducer apparatus of Embodiment 1, further comprising at least one layer of conductive adhesive material or hydrogel located on a front face of the anisotropic material layer.

### Embodiment 12B

The transducer apparatus of Embodiment 12, wherein the conductive adhesive material or hydrogel covers the hole of the anisotropic material layer.

### Embodiment 12C

The transducer apparatus of Embodiment 12, wherein the conductive adhesive material or hydrogel does not cover the hole of the anisotropic material layer.

### Embodiment 13

The transducer apparatus of Embodiment 1, further comprising a substrate for holding the array of electrodes against the subject's body, wherein an outer perimeter of the substrate extends beyond an outer edge of the anisotropic material layer and is contoured to match a shape of the outer edge of the anisotropic material layer.

### Embodiment 13A

The transducer apparatus of Embodiment 13, wherein the substrate covers the hole of the anisotropic material layer.

### Embodiment 13B

The transducer apparatus of Embodiment 13, wherein the substrate partially covers but not entirely covers the hole of the anisotropic material layer.

### Embodiment 13C

The transducer apparatus of Embodiment 13, wherein the substrate does not cover the hole of the anisotropic material layer.

### Embodiment 13D

The transducer apparatus of Embodiment 13, wherein when viewed from the back face of the anisotropic material layer, the substrate comprises a hole, wherein the hole of the substrate has a same shape or a substantially same shape as the hole of the anisotropic material layer.

### Embodiment 13E

The transducer apparatus of Embodiment 13D, wherein when viewed from the back face of the anisotropic material layer, the hole of the substrate is smaller than the hole of the anisotropic material layer.

### Embodiment 13F

The transducer apparatus of Embodiment 13D, wherein when viewed from the back face of the anisotropic material layer, the hole of the substrate matches a size, a shape, and a location of the hole of the anisotropic material layer.

### Embodiment 14

The transducer apparatus of Embodiment 1, further comprising one or more printed circuit board connectors electrically connecting the electrode elements, wherein neither the electrode elements nor the one or more printed circuit board connectors pass over the hole of the anisotropic material layer.

### Embodiment 14A

The transducer apparatus of Embodiment 14, wherein the one or more printed circuit board connectors are positioned equidistantly from the hole of the anisotropic material layer.

### Embodiment 15

The transducer apparatus of Embodiment 1, wherein the array of electrodes has at least two electrode elements and at most six electrode elements.

### Embodiment 16

The transducer apparatus of Embodiment 15, wherein the array of electrodes has only three electrode elements.

### Embodiment 17

The transducer apparatus of Embodiment 1, wherein the anisotropic material layer has different thermal and/or electrical conductivities in a direction perpendicular to the front face of the anisotropic material layer than in directions that are parallel to the front face of the anisotropic material layer.

### Embodiment 18

The transducer apparatus of Embodiment 1, wherein the electrode elements are positioned in existing electrode positions arranged around a centroid of the array, wherein the transducer apparatus further comprises at least one void space in the array of electrodes capable of enclosing an areal footprint equivalent to at least a portion of an areal footprint of at least one existing electrode position and superimposable on at least a portion of at least one existing electrode position by rotation of the array around the centroid.

### Embodiment 18A

The transducer apparatus of Embodiment 18, wherein the electrode elements are positioned in existing electrode positions arranged around a centroid of the array in Cx rotational symmetry, where x is an integer greater than or equal to 3.

### Embodiment 19

The transducer apparatus of Embodiment 18, wherein the at least one void space in the array is capable of enclosing an areal footprint equivalent to at least 40% of an areal footprint of at least one existing electrode position and superimposable on at least 40% of at least one existing electrode position by rotation of the array around the centroid.

### Embodiment 19A

The transducer apparatus of Embodiment 28, wherein the at least one void space in the array is capable of enclosing an areal footprint equivalent to at least 90% or at least 95% of an areal footprint of at least one existing electrode position, and superimposable on at least 90% or at least 95% of at least one existing electrode position by rotation of the array around the centroid.

### Embodiment 20

The transducer apparatus of Embodiment 1, wherein the anisotropic material layer comprises graphite.

### Embodiment 21

The transducer apparatus of Embodiment 1, wherein the anisotropic material layer comprises a sheet of graphite.

### Embodiment 22

The transducer apparatus of Embodiment 1, wherein the anisotropic material layer comprises pyrolytic graphite, graphitized polymer, or graphite foil made from compressed high purity exfoliated mineral graphite.

### Embodiment 23

The transducer apparatus of Embodiment 1, wherein the anisotropic material layer is electrically coupled to the array of electrodes and is located on the front face of the array of electrodes.

### Embodiment 24

A transducer apparatus for delivering tumor treating fields to a subject's body, the transducer apparatus comprising: an array of electrodes, the array configured to be positioned over the subject's body with a front face of the array facing the subject's body, the array comprising electrode elements; an anisotropic material layer electrically coupled to the array of electrodes and located on a front side of the front face of the array, the anisotropic material layer comprising a front face and a back face, the back face facing the array of electrodes; and a foam layer coupled to the front face of the anisotropic material layer, wherein when viewed from the front face of the anisotropic material layer, the anisotropic material layer further comprises a hole centrally located or substantially centrally located in the anisotropic material layer.

### Embodiment 25

The transducer apparatus of Embodiment 24, wherein when viewed from the front face of the anisotropic material layer, the hole is in, or substantially in, a center or a middle of the anisotropic material layer.

### Embodiment 26

A transducer apparatus for delivering tumor treating fields to a subject's body, the transducer apparatus comprising: an array of electrodes, the array configured to be positioned over the subject's body with a front face of the array facing the subject's body, the array comprising electrode elements; an anisotropic material layer electrically coupled to the array of electrodes and located on a front side of the front face of the array, the anisotropic material layer comprising a front face and a back face, the back face facing the array of electrodes, the anisotropic material layer further comprising a hole passing through the front face and the back face of the anisotropic material layer; and a foam layer coupled to the front face of the anisotropic material layer; wherein when viewed from the front face of the anisotropic material layer, the electrode elements of the array of electrodes are arranged in an alternating pattern of electrode elements and blank spaces.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A transducer apparatus for delivering tumor treating fields to a subject's body, the transducer apparatus comprising:
an array of electrodes, the array configured to be positioned over the subject's body with a front face of the array facing the subject's body, the array comprising electrode elements;
an anisotropic material layer electrically coupled to the array of electrodes and located on a front side of the front face of the array, the anisotropic material layer comprising a front face and a back face, the back face facing the array of electrodes, the anisotropic material layer further comprising a hole passing through the front face and the back face of the anisotropic material layer; and
a foam layer coupled to the front face of the anisotropic material layer and covering at least the hole on the front face of the anisotropic material layer.

2. The transducer apparatus of claim 1, wherein when viewed from the front face of the anisotropic material layer, the foam layer covers at most 50% of the area of the anisotropic material layer.

3. The transducer apparatus of claim 1, wherein when viewed from the front face of the anisotropic material layer, the foam layer comprises a hole covering portion to cover the hole on the front face of the anisotropic material layer and the hole covering portion is larger than the hole on the front face of the anisotropic material layer.

4. The transducer apparatus of claim 1, wherein when viewed from the front face of the anisotropic material layer, the foam layer comprises a hole covering portion to cover the hole on the front face of the anisotropic material layer and the foam layer further comprises a perimeter portion covering a perimeter of the anisotropic material layer.

5. The transducer apparatus of claim 4, wherein when viewed from the front face of the anisotropic material layer, the foam layer further comprises at least one connecting portion connecting the hole covering portion and the perimeter portion, wherein the at least one connecting portion covers the anisotropic material layer.

6. The transducer apparatus of claim 1, wherein when viewed from the back face of the anisotropic material layer, a perimeter of the foam layer is larger than a perimeter of the anisotropic material layer.

7. The transducer apparatus of claim 6, wherein when viewed from the back face of the anisotropic material layer, a shape of the perimeter of the foam layer is the same as or substantially the same as a shape of the perimeter of the anisotropic material layer.

8. The transducer apparatus of claim 1, wherein the hole is centrally located in the front face and the back face of the anisotropic material layer.

9. The transducer apparatus of claim 1, wherein the hole is circular, oval, ovoid or substantially circular, oval or ovoid in shape.

10. The transducer apparatus of claim 1, wherein the electrode elements are positioned around the hole of the anisotropic material layer.

11. The transducer apparatus of claim 1, wherein the electrode elements are positioned equidistantly from each other and equidistantly from the hole of the anisotropic material layer.

12. The transducer apparatus of claim 1, further comprising at least one layer of conductive adhesive material or hydrogel located on a front facing side of the anisotropic material layer.

13. The transducer apparatus of claim 1, further comprising a substrate for holding the array of electrodes against the subject's body, wherein an outer perimeter of the substrate extends beyond an outer edge of the anisotropic material layer and, optionally, is contoured to match a shape of the outer edge of the anisotropic material layer.

14. The transducer apparatus of claim 1, further comprising one or more printed circuit board connectors electrically connecting the electrode elements, wherein neither the electrode elements nor the one or more printed circuit board connectors pass over the hole of the anisotropic material layer.

15. The transducer apparatus of claim 1, wherein the array of electrodes has at least two electrode elements and at most six electrode elements.

16. The transducer apparatus of claim 15, wherein the array of electrodes has only three electrode elements.

17. The transducer apparatus of claim 1, wherein the anisotropic material layer has different thermal and/or electrical conductivities in a direction perpendicular to the front face of the anisotropic material layer than in directions that are parallel to the front face of the anisotropic material layer.

18. The transducer apparatus of claim 1, wherein the electrode elements are positioned in existing electrode positions arranged around a centroid of the array, wherein the transducer apparatus further comprises at least one void space in the array of electrodes capable of enclosing an areal footprint equivalent to at least a portion of an areal footprint of at least one existing electrode position and superimposable on at least a portion of at least one existing electrode position by rotation of the array around the centroid.

19. The transducer apparatus of claim 18, wherein the at least one void space in the array is capable of enclosing an areal footprint equivalent to at least 40% of an areal footprint of at least one existing electrode position and superimposable on at least 40% of at least one existing electrode position by rotation of the array around the centroid.

20. The transducer apparatus of claim 1, wherein the anisotropic material layer comprises graphite.
